(19) United States
(12) Patent Application Publication
(10) Pub. No.: US 2023/0077040 A1
(43) Pub. Date: Mar. 9, 2023

(54) NONSURGICAL METHODS FOR NASAL MODIFICATION INCLUDING NASAL TIP ELEVATION AND/OR ELONGATION

(71) Applicant: Arthur Yajing YU, La Canada Flintridge, CA (US)

(72) Inventor: Arthur Yajing YU, La Canada Flintridge, CA (US)

(21) Appl. No.: 17/502,744

(22) Filed: Oct. 15, 2021

Related U.S. Application Data
(60) Provisional application No. 63/236,978, filed on Aug. 25, 2021.

(51) Int. Cl. A61F 2/18; A61B 17/34

(52) U.S. Cl. CPC A61F 2/186; A61B 17/3468

(57) ABSTRACT

Nonsurgical methods of modifying at least a portion of a nose of a subject, such as a tip of the nose of the subject, are provided herein. The method can include performing one or more of a first injection of a first filler composition and a second injection of a second filler composition. The first injection includes depositing the first filler composition in a first region defined as a septal space behind a middle crura and a medial crura of the nose, substantially level and/or above an anterior nasal spine of the nose, and in front of a caudal septal cartilage of the nose. The second injection includes depositing the second filler composition in a second region defined as the septal space behind the middle crura and the medial crura of the nose substantially level with the footplates of the nose and the columella segment of the medial crura of the nose.

### CROSS-REFERENCE TO RELATED APPLICATIONS

This application claims the benefit of and priority to U.S. Provisional Application No. 63/236,978, filed on 25 Aug. 2021, the entire contents of which are hereby incorporated herein by reference.

### FIELD

The present invention relates to nonsurgical methods for modifying at least a portion of a nose of a subject, such as a tip of the nose, using one or more filler compositions.

### BACKGROUND

The nose, being in the middle of the face, functions as the centerpiece that coordinates all other facial features including the eyes, the brows, the lips, and the chin. Although the eyes command peoples' attention on a frontal view, the nose may become the focal point when a person's profile is viewed. FIGS. 1A and 1B provide a detailed structural anatomy of a human nose.

Before the availability of fillers, surgical rhinoplasty was the most frequently utilized approach for nose modification or enhancement. However, even when using rib cartilages for nasal surgeries, complications, including malformation, warping and displacement, as well as donor-site morbidities are common, and the re-operation rate can be as high as 5% to 12% and may even be higher. Furthermore, even if surgeons are facile with their techniques, patients may not be satisfied with their results, due to the stiffness and the immobility of the resulting nose tips.

As technologies have developed, individuals are less likely to seek surgical correction of their noses. Instead, they are more likely to seek simple and natural enhancements. In this regard, filler rhinoplasty or nonsurgical rhinoplasty has become a more desired choice. The materials used for nonsurgical rhinoplasty have evolved from paraffin and liquid silicone to the more promising hyaluronic acid gel and calcium hydroxyapatite. Because of the seemingly ease of filler application, the nonsurgical rhinoplasty can be performed not only by core specialty physicians, but also by non-core physicians and nurses alike.

Presently, various fillers approved by FDA have been used in correction of nose deformities, including deformities of the radix, dorsum, nasal base, nasal alar, and the nasal tip. However, the nasal tip enhancement remains an elusive target due to many limitations, such as effectiveness, safety issues and technical difficulties.

As far as the tip injection techniques are concerned, many practitioners inject either from the supra-tip lobule or the infra-tip lobule for tip improvement, by piling up the fillers above the tip cartilages. Unfortunately, these maneuvers can cause two major problems. One problem is possible vascular compromise. The second problem is widening of the nasal tip with a bulbous appearance, which becomes more obvious with time passing, especially when a Vycross® filler is used. Furthermore, as illustrated in FIG. 2A, a ratio of infra-tip lobule 20 length (a) to columella 30 length (b), may be compromised, with the piling on of fillers over the tip cartilages. Ideally, the infra-tip lobule length to columella length ratio should be close to 1:2, as shown in FIG. 2D, as opposed to the converse ratio as shown in both FIGS. 2A and 2B.

In a series of 5000 nose injections, Harb et al. injected filler through nose tip skin onto the anterior surface of the cartilages. Harb, A. et al., The Nonsurgical Rhinoplasty: A Retrospective Review of 5000 Treatments. Plast Reconstr Surg 2020; 145: 661-667. While the results were relatively good, there were 24 arterial occlusions and 2 infections, consistent with the notion that the tip superficial layers is a dangerous place for injection induced vascular compromises.

Bertossi et al., did a prospective study on filler rhinoplasty in 150 patients. Bertossi, D. et al., Nonsurgical Rhinoplasty: Nasal Grid Analysis and Nasal Injecting Protocol. Plast Reconstr Surg 2018; 143:428-439. In the study, a first injection of the anterior nasal spine was performed, followed by defining the tip. The results showed modest tip elevation. In another prospective study by Liew et al., 29 patients were treated with a set algorithm, using Voluma®, encompassing the whole nose. Liew, S. et al., Efficacy and Safety of a Hyaluronic Acid Filler to Correct Aesthetically Detracting or Deficient Features of the Asian Nose: A Prospective, Open-Label, Long-Term Study. Aesth Surg J 2016; 36:760-772. The filler was injected into the columella. However, the cases exhibited very limited tip improvement. More effective improvement was shown by Segreto et al. Segreto, F. et al., Nonsurgical Rhinoplasty: A Graft-based Technique. Plast Reconstr Surg Glob Open 2019; 7:e2241. The combination of anterior nasal spine and tip injection with Juvederm 4® did show better corrections. The Eifel Tower Nose-Lift® takes a very similar approach. Nonetheless, the tip improvement remains limited in the presented cases.

Dr. Jung used Teosyal® filler for Asian noses by injecting the filler on the supra-cartilaginous plane over the columella to the anterior nasal spine. Jung, G. S. Filler rhinoplasty based on anatomy: The dual plane technique. JPRAS Open. 2019; 20:94-100. While the patients did have positive results on dorsal improvement, the tip improvement was barely noticeable. Youn et al. presented more detailed measurements on filler rhinoplasty with a similar method. Youn, S. H. et al., Filler Rhinoplasty Evaluated by Anthropometric Analysis. Dermatol Surg 2016; 42:1071-1081. After the anterior nasal spine was augmented, a “columella strut” was created with filler injection. Reasonable tip elevation with modest tip rotation was achieved.

Coimbra et al. described tip augmentation using a needle with Juvederm® Volift®, either from the nasolabial angle up or through the nasal tip down. Coimbra D D, de Oliveira B S, and Caballero Uribe N C. Nasal filling with a new hyaluronic acid: a series of 280 cases. Surg Cosmet Dermatol 2015; 7:320-326. The results were relatively good but still not ideal, probably due to too small a volume injected (0.1 to 0.2 cc) or the injection being too close to the columella. Bravo et al. treated 44 patients using a 22G cannula to inject through the nasal tip skin, reaching the lower part of the columella. Bravo, B. S. F. et al., Evaluation and Proportion in Nasal Filling with Hyaluronic Acid. J Clin Aesthet Dermatol 2018; 11:36-40. Again, the results were suboptimal, most likely due to the injection proximity to the columella.

The suboptimal results for nose tip improvement are mainly due to the lack of sufficient understanding of human nasal anatomy. In most of the peer reviewed publications, the failure can be further subcategorized into the following: 1) piling up of fillers by directly injecting into the nose tip, intra-dermally, subdermally, subcutaneously, intra-muscularly, sub-muscularly, sub-superficial musculoaponeurotic system (SMAS), over the peri-chondrial membrane of the cartilages, etc., making the tip bulbous and in most cases, compromise the infra-tip lobule to columella ratio, leading to worsening of the nasal aesthetics; 2) injecting filler directly in between the columella cartilages in an effort to make a columella strut, for propping up of the nose tip, which results in tying up of the columella, as a result of the filler and surrounding fibrous tissue inter-play, which, in turn, tethers the columella cartilages preventing the columella and, therefore, the nose tip from being able to lift up, as intended; and 3) injecting directly into the surface of the nose anterior nasal spine, in an effort to provide more support to the tip from below. But even with additional injection(s) in between or near the columella structures, the nose tip lift is still sub-optimal. Thus, there is a need for improved nonsurgical methods for modifying a nose of a subject, for example, for elongating and/or elevating a tip of the nose of the subject with one or more filler compositions.

### SUMMARY OF THE INVENTION

This section provides a general summary of the disclosure and is not a comprehensive disclosure of its full scope or all of its features.

Provided herein are nonsurgical methods of modifying at least a portion of a nose of a subject. In one embodiment, the method may include performing one or more of a first injection of a first filler composition and second injection of a second filler composition. The first injection includes depositing the first filler composition in a first region defined as a septal space behind a middle crura and a medial crura of the nose, substantially level and/or above an anterior nasal spine of the nose, and in front of a caudal septal cartilage of the nose. The second injection includes depositing the second filler composition in a second region defined as the septal space behind the middle crura and the medial crura of the nose and substantially level with footplates of the nose and the columella segment of the medial crura of the nose. The second region may be located more posterior to the first region. The first filler composition and the second filler composition may be the same or different.

Other embodiments, including particular aspects of the embodiments summarized above, will be evident from the detailed description that follows.

### BRIEF DESCRIPTION OF THE DRAWINGS

The drawings described herein are for illustrative purposes only of selected embodiments and not all possible implementations and are not intended to limit the scope of the present disclosure.

FIG. 1A illustrate a frontal view of the anatomical structure of a nose.

FIG. 1B illustrates a lateral view of the anatomical structure of a nose.

FIG. 2A is an image of a subject after treatment with a prior nasal modification method. FIGS. 2B-2D are illustrations of a subject before treatment (FIG. 2C), after treatment with a prior nasal modification method (FIG. 2B), and after treatment with a nasal modification method according to the present disclosure (FIG. 2D).

FIGS. 3A and 3B illustrate a nasal modification method according to the present disclosure.

FIGS. 4A-4D illustrate a nasal modification method according to the present disclosure.

FIG. 5A are images of a subject before and after treatment with a nasal modification method according to the present disclosure.

FIG. 5B are images of a subject before and after treatment with a nasal modification method according to the present disclosure from a worm's eye view.

FIG. 6 is an illustration of a nasal modification method according to the present disclosure.

FIG. 7 are images of a subject before and after treatment with a nasal modification method according to the present disclosure.

FIG. 8 are images of a subject before and after treatment with a nasal modification method according to the present disclosure.

FIGS. 9A and 9B are images of a subject before and after treatment with a nasal modification method according to the present disclosure.

FIG. 10 are images of a subject before and after treatment with a nasal modification method according to the present disclosure.

FIG. 11 are images of a subject before and after treatment with a nasal modification method according to the present disclosure.

FIG. 12 are images of a subject before and after treatment with a nasal modification method according to the present disclosure.

FIG. 13 are images of a subject before and after treatment with a nasal modification method according to the present disclosure.

FIGS. 14A and 14B are images of a subject before and after treatment with a nasal modification method according to the present disclosure.

FIG. 15 are images of a subject before and after treatment with a nasal modification method according to the present disclosure.

FIGS. 16A and 16B are images of a subject before and after treatment with a nasal modification method according to the present disclosure.

FIG. 17 are images of a subject before and after treatment with a nasal modification method according to the present disclosure.

FIG. 18 are images of a subject before and after treatment with a nasal modification method according to the present disclosure.

FIG. 19 are images of a subject before and after treatment with a nasal modification method according to the present disclosure.

FIG. 20 are images of a subject before and after sequential treatment with a nasal modification method according to the present disclosure.

FIG. 21 are images of a subject before and after sequential treatment with a nasal modification method according to the present disclosure.

FIG. 22 are images of a subject before and after sequential treatment with a nasal modification method according to the present disclosure.

FIG. 23 are images of a subject before and after sequential treatment with a nasal modification method according to the present disclosure.

FIG. 24 are images of a subject before and after treatment with a nasal modification method according to the present disclosure.

FIG. 25 are images of a subject before and after treatment with a nasal modification method according to the present disclosure.

FIG. 26 are images of a subject before and after treatment with a nasal modification method according to the present disclosure.

FIG. 27 are images of a subject before and after treatment with a nasal modification method according to the present disclosure.

FIG. 28 are images of a subject before and after treatment with a nasal modification method according to the present disclosure.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

Example embodiments will now be described more fully with reference to the accompanying drawings.

Nonsurgical methods of modifying at least portion of a nose of a subject are provided herein. A method may include performing a first injection of a first filler composition, a second injection of second filler composition, or both the first injection and the second injection. The first injection may elevate a nose tip and the second injection may elongate a nose tip. The first filler composition and the second filer composition may be deposited with a needle or a cannula of any suitable size. For example, a 27 G, ½-inch needle may be used for subjects, such as Asian and African American subjects, and a 27 G, 1-inch cannula may be used for subjects, such as Hispanic and Caucasian subjects. Or in situations where required, the use of a needle or a cannula of any length and of any size can be used in a subject of any race.

Additionally, the first injection, the second injection, or both may include a retro-injection or an antegrade injection. A “retro-injection” refers to the depositing or ejection of the filler composition from a needle or a cannula as the needle or cannula is being removed or withdrawn from the nose, for example, from the septal space of the nose. An “antegrade injection” refers to the depositing or ejection of the filler composition from a needle or a cannula as the needle or cannula is entering the nose, for example, entering into the septal space of nose.

In any embodiment, prior to the first and/or second injection(s), any suitable topical anesthetic (e.g., lidocaine, pramoxine, etc.) may be applied to the area(s) surrounding the injection site of the subject. After a suitable amount of time, for example, about 5 to 25 minutes or more, the injection site and surrounding areas may undergo ice packing, for example, for about 2 minutes. Following skin disinfection, the first and/or second injection(s) may be performed.

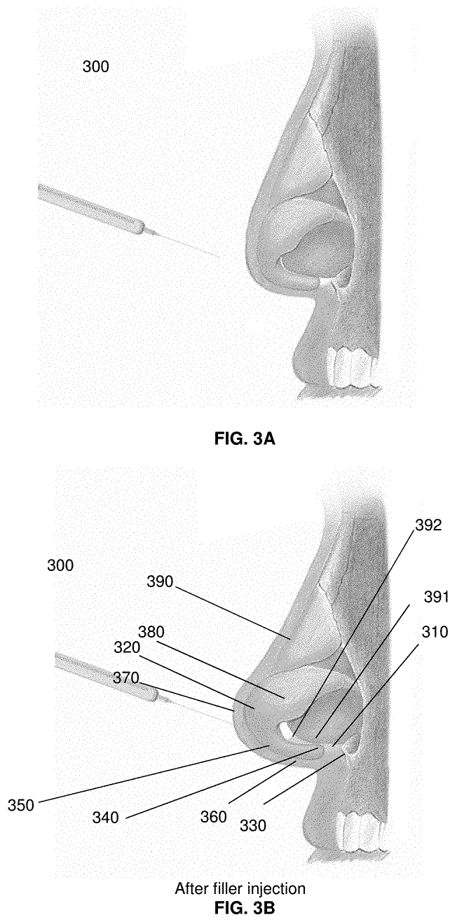

FIG. 3A illustrates a nose 300 prior to a first injection and FIG. 3B illustrates the nose 300 after the first injection. In any embodiment, as illustrated in FIG. 3B, the first injection includes depositing the first filler composition 310 in a first region defined as a septal space behind a middle crura 320 and a medial crura 350 of the nose, substantially level and/or above an anterior nasal spine 330 of the nose, and in front of or anterior to a caudal septal cartilage 391 of the nose. The first region, which includes the first filler composition 310, may be further defined as below and/or level with the footplates 340 of the nose and at a columella segment of medial crura 350 of the nose, for example, at a midpoint of the columella segment of the medial crura 350. In some embodiments, the first filler composition may not be deposited above a mid-level or midpoint 392 of the medial crura 350 because, in some instances, depositing the first filler composition above the mid-level or midpoint of the medial crura 350 may result in the first filler composition bleeding through the scroll area onto the upper lateral cartilage 390 causing deformation of the nasal dorsum.

During the first injection, the needle or cannula may enter the nose 300, for example, at the mid-point cephalic to the middle crura 320 of the lower lateral cartilage 380, travel down to a mid-point between the columella 360 and the caudal septal cartilage (not shown) reaching the anterior nasal spine 330. The needle or cannula may travel down the potential space (septal space) in the middle between an anterior/caudal edge of the septal cartilage and the medial crura 350. After aspiration, a retro-injection may be carried out to deposit the first filler composition, stopping at the mid-level of the medial crura 350. Additionally or alternatively, the first filler composition may be deposited prior to the needle or cannula reaching the anterior nasal spine 330. It is also contemplated herein that an antegrade injection may be used during the first injection. In any embodiment, the first filler composition may not be deposited in a surface of a tip 370 of the nose.

In any embodiment, the first injection may further include holding the columella 360 and/or nasal tip 370 (also referred to as tip 370) of the nose in a first direction in order to extend open the septal space before, while, and/or after the first injection is performed. The first direction may be in an upward direction, a caudal direction, a rotated direction, or a combination thereof. For example, in order to provide an elevated nose tip and/or cephalic rotation, the columella 360 and/or nasal tip 370 may be held in an upward direction before, while, and/or after the first injection is performed. The injector may use the thumb and the middle fingers to hold the columella 360 while pulling slightly upward. Additionally or alternatively, the nasal tip 370 and columella 360 may be pressed against anterior septal cartilage of the nose to elevate the nasal tip 370. During the first injection, the septum structure may be distended and the nasal tip 370 can be elevated. The nasal tip structure itself may not be altered by the first filler composition because the first filler composition is not directly deposited on the nose tip, but the nasal tip does become elevated due to the lifting of the whole nasal tip structure. Additionally, the shape of the nasal tip does become better defined with a better shape as a result of the compression on it by the surrounding skin and tissue.

Additionally or alternatively, the first direction may be a caudal direction or downward and a tip of the nose may be elevated, elongated, or both. Additionally or alternatively, the first direction may be both the upward direction and the rotated direction and a tip of the nose and/or the nose may be shortened due to the nasal tip 370 undergoing rotation. It is also contemplated herein that if the nasal tip undergoes undesirable rotation, the columella 360 and/or nasal tip 370 may be held only in the caudal direction to offset the rotation.

Alternatively, the first injection may not further include holding the columella 360 and/or nasal tip 370 of the nose in a first direction.

In any embodiment, a nasal tip 370 may be elevated or lifted by greater than zero (0) mm, greater than or equal to about 0.25 mm, greater than or equal to about 0.5 mm, greater than or equal to about 0.95 mm, greater than or equal to about 1 mm, greater than or equal to about 2 mm, greater than or equal to about 3 mm, greater than or equal to about 4 mm, greater than or equal to about 5 mm, greater than or equal to about 6 mm, greater than or equal to about 7 mm, greater than or equal to about 8 mm, greater than or equal to about 9 mm, or about 10 mm; or from greater than zero mm to about 10 mm, greater than zero mm to about 8 mm, greater than zero mm to about 5 mm, greater than zero mm to about 3 mm, greater than zero mm to about 1 mm, about 2 mm to about 10 mm, about 2 mm to about 8 mm, about 2 mm to about 5 mm, or about 2 mm to about 4 mm. Alternatively, the nasal tip 370 may not be elevated or lifted, i.e., the nasal tip 370 may be elevated by zero mm. In order to measure nasal tip elevation, a first nasal tip height is measured prior to the first injection and/or the second injection, then a second nasal tip height is measured after the first injection and/or the second injection, and the difference between the first and second nasal tip heights is the nasal tip elevation. Nasal tip height is a measure of the highest point of the nasal tip (see FIG. 3A nasal tip 370) from the nasolabial angle.

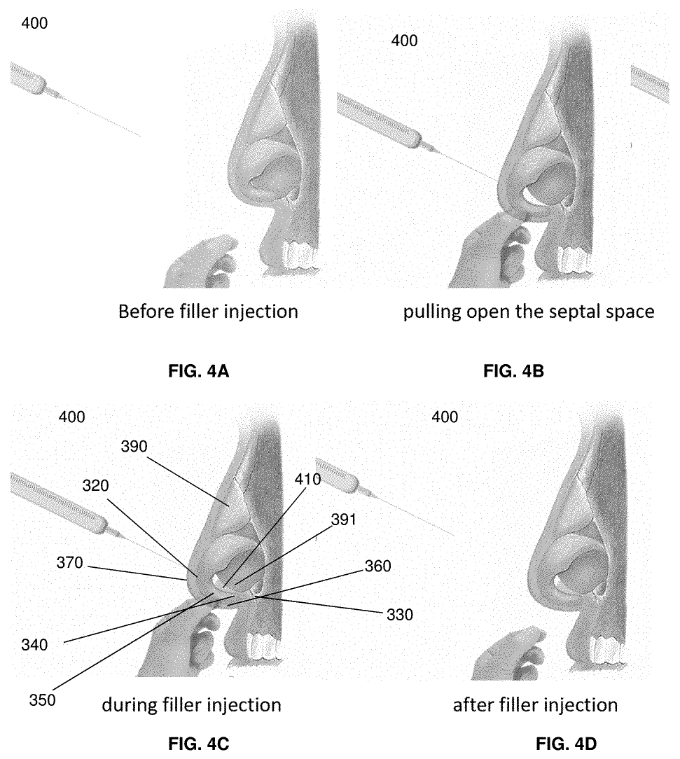

With regard to the second injection, FIGS. 4A and 4B illustrate a nose 400 prior to a second injection, FIG. 4C illustrates the nose 400 during the second injection, and FIG. 4D illustrates the nose 400 after the second injection. In any embodiment, as illustrated in FIGS. 4C and 4D, the second injection includes depositing the second filler composition 410 in a second region defined as a septal space behind a middle crura 320 and medial crura 350 of the nose and substantially level with and/or above the footplates 340 of the nose and the columella segment of the medial crura 350 of the nose. The second region may be located more posterior to the first region. The caudal septal cartilage 391 comprises a posterior part of this septal space including the second filler composition 410. For the second injection, an entry point of the needle or cannula may be positioned more posteriorly in front of the caudal septal cartilage 391 compared to the first injection and closer to and/or embracing the caudal septal cartilage.

During the second injection, the needle or cannula may enter the nose 400, for example, through the nasal tip 370 above/cephalic to (e.g., about 2 to 5 mm above/cephalic to) the entry of the needle or cannula for the first injection, at a point of the anterior/caudal edge of the septal cartilage. The needle or cannula may travel down, going down the anterior edge of the septal cartilage aiming toward the anterior nasal spine 330, but without reaching the anterior nasal spine 330. After aspiration, a retro-injection may be carried out to deposit the second filler composition, stopping at a mid-level of the caudal septal cartilage 391 to avoid leaking through the scroll area onto the surface of the upper lateral cartilages 360. The second filler composition 410 may be deposited mainly at the footplate 340 and the columella segment of the medial crura 350. Additionally or alternatively, the second filler composition 410 may be deposited in front of the anterior nasal spine 330. After the second injection, the septal space structures may be distended and remain extended.

In any embodiment, the second injection may further include holding the columella 360 and/or nasal tip 370 (also referred to as tip 370) of the nose in a second direction in order to extend open the septal space before, while, and/or after the second injection is performed. The second direction may be of a caudal direction, an outward direction, an upward direction, or a combination thereof. For example, in order to provide an elongated nose tip, the columella 360 and/or nasal tip 370 may be held in an outward and/or caudal direction before, while, and/or after the second injection is performed. The injector may use the thumb and the middle fingers to hold the columella 360 while pulling away from the subject's head (e.g., in a caudal direction and/or outward direction). Additionally or alternatively, the nasal tip 370 and columella 360 may be held in an upward direction to elevate the nasal tip 370. After the second injection, the septal space between the septal cartilage and the medial crura may be inflated, and the whole nasal tip structure can extend in the sagittal plane, causing the nose tip to move either caudally (tip elongation) or anteriorly (tip elevation), or both, as directed by the injector's fingers. This effect mimics that of a bounce house, in which no column is present, yet the whole structure is easily bolstered up with the introduction of air.

Alternatively, the second injection may not further include holding the columella 360 and/or nasal tip 370 of the nose in a second direction.

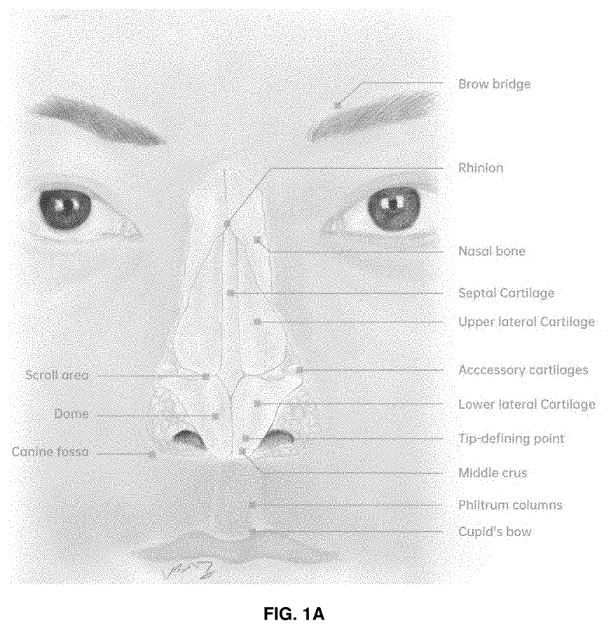

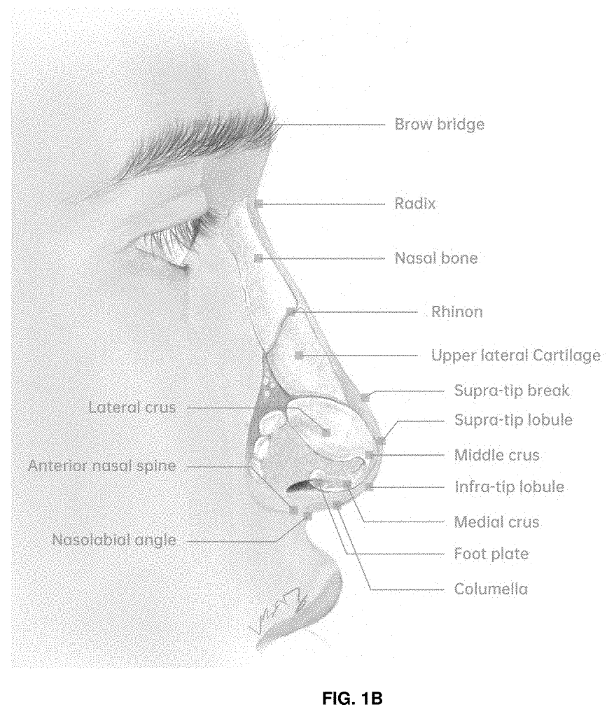

In any embodiment, a nasal tip 370 may be elongated or lengthened by greater than zero (0) mm, greater than or equal to about 0.25 mm, greater than or equal to about 0.5 mm, greater than or equal to about 0.95 mm, greater than or equal to about 1 mm, greater than or equal to about 2 mm, greater than or equal to about 3 mm, greater than or equal to about 4 mm, greater than or equal to about 5 mm, greater than or equal to about 6 mm, greater than or equal to about 7 mm, greater than or equal to about 8 mm, greater than or equal to about 9 mm, or about 10 mm; or from greater than zero mm to about 10 mm, greater than zero mm to about 8 mm, greater than zero mm to about 5 mm, greater than zero mm to about 3 mm, greater than zero mm to about 1 mm, about 2 mm to about 10 mm, about 2 mm to about 8 mm, about 2 mm to about 5 mm, or about 2 mm to about 4 mm. Alternatively, the nasal tip 370 may not be elongated or lengthened, i.e., the nasal tip 370 may be elongated by zero mm. In order to measure nasal tip elongation, a first nasal tip length is measured prior to the first injection and/or the second injection, then a second nasal tip length is measured after the first injection and/or the second injection, and the difference between the first and second nasal tip lengths is the nasal tip elongation. The nasal tip length is measure by identifying and/or marking the rhinion (FIGS. 1A and 1C) and nose tip via the nose tip defining points (FIG. 1A) and measuring the distance between the nose tip and the rhinion.

In any embodiment, the first filler composition 310 and the second filler composition 410 may be deposited into the septal space of the nose 300, 400. For example, the first filler composition 310 and the second filler composition 410 may be deposited anywhere in the nasal septal space from the anterior nasal spine 330 to the columella 360, to the upper space behind the medial crura and to a tip part of the nasal tip cartilage.

However, the first filler composition 310 and the second filler composition 410 may not be directly deposited in between or close to the columella structure because it is believed that mimicking a columella strut with the filler does not work well for nose tip lifting. As far as vascular compromise is concerned, the injection site of the first and second filler compositions is far from the confluence of the six major blood supplies (the lateral nasal branch from angular arteries, columellar arteries, and nasal dorsal arteries) at the superficial layer (above the superficial musculoaponeurotic system, SMAS) of the tip. Hence, the methods described herein are safer.

In any embodiment, both the first injection and the second injection may be performed or stacked (first injection followed by the second injection and vice versa), for example, for a subject with short and flat nasal tips, thus achieving nasal tip elevation and elongation. Additionally or alternatively, the first injection, the second injection, or both may be performed individually each one, more than once or sequentially, for example, for a subject with substantial deficiency in the tip and canine fossa region. For example, the first and/or second injections may each be performed once followed by a period of time (e.g., 1 week, 2 weeks, 3 weeks, 1 month, 3 months, 6 months, 9 months, 1 year, etc.) and then performed again followed by the period of time, and so on, until the desired result is achieved.

In any embodiment, the first filler composition and the second filler composition may each comprise hyaluronic acid (e.g., Vycross® fillers, such as Juvederm® fillers, nonanimal stabilized hyaluronic acid (NASHA) fillers, such as Restylane® fillers, or any other hyaluronic acid fillers), calcium hydroxyapatite (e.g., Radiesse® fillers and any other brands or similar compositions), fat (autologous or heterologous fat), soft tissue of any origin, cartilage or bone products of any origin, agar, agarose, collagen, dextran beads, polymethyl methacrylate (PMMA), methyl methacrylate, polylactic acid, bone powder of any origin, polydioxanone (PDO) powder, PDO gel or any other form, silicone of any physical form, polytetrafluoroethylene (PTFE) in any form, expanded polytetrafluoroethylene (ePTFE), a polyalkylimide (e.g., hydrophilic polyacrylamide gel, such as Amazing Gel), silk in any form, any other human tissue (e.g., dermal tissue, AlloDerm® powders), animal tissue (e.g., porcine tissue, such as STRATTICE™ Reconstructive Tissue Matrix), plant substrate in any form (e.g., CollPlant substrates), any synthetic material, or any other suitable filler material, or a combination thereof. In some embodiments, the first filler composition and the second filler composition each may be a nonanimal stabilized hyaluronic acid (NASHA), a highly cross-linked non-animal hyaluronic acid gel (Vycross® fillers), a hyaluronic acid gel of more cross-linking, a hyaluronic acid gel of less cross-linking, a hyaluronic gel of animal origin, a cross-linked nonanimal derived hyaluronic acid gel, a hyaluronic acid product without any cross-linking, or a combination thereof. The first filler composition and the second filler composition may be the same or different.

The methods described herein allow for depositing the first filler composition and the second filler composition in smaller or larger amounts. For example, an amount of each of the first filler composition deposited and the second filler composition deposited may be greater than or equal to about 0.01 cm³, greater than or equal to about 0.05 cm³, greater than or equal to about 0.1 cm³, greater than or equal to about 0.25 cm³, greater than or equal to about 0.5 cm³, greater than or equal to about 0.75 cm³, greater than or equal to about 1 cm³, greater than or equal to about 1.25 cm³, greater than or equal to about 1.5 cm³, greater than or equal to about 1.75 cm³, or about 2 cm³; or from about 0.01 cm³ to about 2 cm³, about 0.01 cm³ to about 1.75 cm³, about 0.01 cm³ to about 1.5 cm³, about 0.01 cm³ to about 1.25 cm³, about 0.01 cm³ to about 1 cm³, about 0.01 cm³ to about 0.75 cm³, about 0.01 cm³ to about 0.5 cm³, about 0.01 cm³ to about 0.25 cm³, about 0.01 cm³ to about 0.1 cm³, about 0.01 cm³ to about 0.05 cm³, about 0.1 cm³ to about 2 cm³, about 0.1 cm³ to about 1.75 cm³, about 0.1 cm³ to about 1.5 cm³, about 0.1 cm³ to about 1.25 cm³, about 0.1 cm³ to about 1 cm³, about 0.1 cm³ to about 0.75 cm³, about 0.1 cm³ to about 0.5 cm³, or about 0.1 cm³ to about 0.25 cm³.

In further embodiments, the methods described herein may also include performing an optional third injection of a third filler composition. The third injection may include depositing the third filler composition in the radix region of the nose, a dorsum region of the nose, or both. The third filler composition may be the same as one or both of the first filler composition and the second filler composition. Alternatively, the third filler composition may be different from both the first filler composition and the second filler composition. The third filler composition may comprise hyaluronic acid (e.g., Vycross® fillers, such as Juvederm® fillers, nonanimal stabilized hyaluronic acid (NASHA) fillers, such as Restylane® fillers, or any other hyaluronic acid fillers), calcium hydroxyapatite (e.g., Radiesse® fillers and any other brands or similar compositions), fat (autologous or heterologous fat), soft tissue of any origin, cartilage or bone products of any origin, agar, agarose, collagen, dextran beads, polymethyl methacrylate (PMMA), methyl methacrylate, polylactic acid, bone powder of any origin, polydioxanone (PDO) powder, PDO gel or any other form, silicone of any physical form, polytetrafluoroethylene (PTFE) in any form, expanded polytetrafluoroethylene (ePTFE), a polyalkylimide (e.g., hydrophilic polyacrylamide gel, such as Amazing Gel), silk in any form, any other human tissue (e.g., dermal tissue, AlloDerm® powders), animal tissue (e.g., porcine tissue, such as STRATTICE™ Reconstructive Tissue Matrix), plant substrate in any form (e.g., CollPlant substrates), any synthetic material, or any other suitable filler material, or a combination thereof.

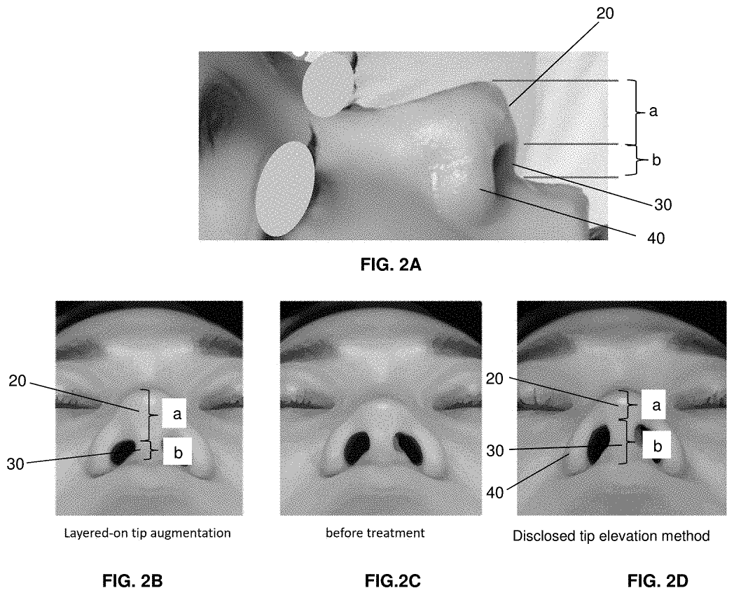

While nasal modifications including nasal tip elevation and elongation are described above, it also contemplated herein that the methods describe herein encompass the following further modifications and improvements: elevating the columella of the nose, elongating the columella of the nose, shortening the tip of the nose, increasing firmness of the nose, narrowing of the dome of the nose, elevating the footplates, decreasing width of an alar of the nose, increasing prominence of philtrum columns, modifying the shape of the nostrils (e.g., increasing oblong shape of the nostrils), or any combination thereof. Additionally or alternatively, the methods described herein may also result in an improved ratio of infra-tip lobule length to columella length. Referring back to FIG. 2D, the methods described herein can result in a more desirably increased columella 30 length (b) which is greater than the lobule (infra-tip lobule) 20 length (a) in contrast to prior methods which piled on of fillers over the tip cartilages, as shown in FIG. 2B. The prior methods resulted in an infra-tip lobule 20 length (a) increase, with possible shortening of the columella 30 length (b). Thus, the methods described herein may result in the subject having a ratio of the infra-tip lobule 20 length (a) to columella 30 length (b) closer to 1:2, a recognized ideal ratio. The results of the methods described herein may last for many months or for years, for example, from 9 months to two years.

Table 1 below summarizes the observed benefits from performing the methods described herein.

TABLE 1
Benefits of filler injection into the septal space between caudal
septal cartilage and columella cartilages. All benefits may
not show in one case or in one treatment session.

| No. | Benefits |
|---|---|
| 1. | Nasal tip elevated and firmer |
| 2. | Nasal tip elongation |
| 3. | Nasal tip could be downturned (with elongation) |
| 4. | Nasal tip rotation (upturn) to shorten a long nose |
| 5. | Nasal dome stands out, and narrower |
| 6. | Finer tip cartilaginous structures |
| 7. | Columella lengthened |
| 8. | Footplates elevated |
| 9. | Infra-tip lobule to columella ratio improved |
| 10. | Nostrils change to oblong in shape |
| 11. | Alar width reduced |
| 12. | Nasal tips do not become bigger, as other injection |
| | techniques would |
| 13. | Philtrum columns become more prominent |
| 14. | Nasolabial Angle and veiling improved |
| 15. | Upper lip shortened |
| 16. | Lip better positioned |
| 17. | Depressor septi nasi function eliminated with better |
| | looking smiles |
| 18. | Gummy smile/labial incompetence improved |
| 19. | Works as a tissue expander—successive injections |
| | generate progressive improvements |
| 20. | Salvages botched nasal implant result, especially with |
| | capsular contracture |
| 21. | Acts as the key in facial profile improvement |
| 22. | It is useful for all races |

The methods described herein advantageously may be useful in treating a subject having one or more of a nasal hump deformity, a saddle nose deformity, a nasal deformity caused by capsular contracture after a nasal implant. Furthermore, the methods described herein may be performed on a subject that is Asian, Caucasian, Hispanic, Arabian, African American, or a combination thereof.

Without being bound by theory, it is believed that the methods described herein are not based on a columella strut effect. Rather, the first and second filler compositions are injected into the septal space, which may be further extended by holding and pulling away the columella and/or nose tip, to obtain a significant elevating and/or elongating effect. The anterior nasal spine need not be reached during the injections with the described methods. To the contrary, if the filler compositions are injected between the medial crura or in their vicinity, the columella may become constrained and the lifting effect may be limited. This constraint on nasal tip lifting may be due to the distension of the columella and surrounding fibrous structures resulting in the “tie up” of the columella per se for mechanisms unknown. However, in the methods described herein, the filler composition distends the potential septal space between the mucosal septum only in the sagittal direction, and the sagittal expansion of the space produces an “inflated bounce house” effect. This may be attributable to the abundance of the dense fibro-fatty tissue network, which tightly holds the membranous septum and the cartilages in a relatively volume-constrained space. Because the septal cartilage is fixed and the columella is not tied up, the “inflated bounce house” effect freely elevates or distends the columella and tip structures.

Lee et al. reports that a significant amount of densely packed elastin, collagen fibers and fatty tissues are present in the space between the caudal septal cartilage and the medial crura. Lee M. R. et al., Soft-Tissue Composition of the Columella and Potential Relevance in Rhinoplasty. Plast Reconstr Surg 2014; 134:621-625. Without being bound by theory, it is believed that it is the cartilaginous framework and supporting soft tissues, together, which create a construct responsible for ultimate tip conduct. It is further suggested that the combined influences of cartilaginous structure and accompanying soft tissue together determine nasal tip projection and position. It is believed that it is the dense fibro-fatty network that forms the foundation for the superior effectiveness of the methods disclosed herein.

Additionally, and without being bound by theory, the disclosed methods' effectiveness may depend on various mechanical effects as follows:
1) The initially deposited filler composition sits on the center of a dense layer of fibro-fatty tissue network, which acts as a “hammock” that supports the filler and with more influx of filler, the filler starts to push the fibro-fatty tissue above and in front of it, upward or caudally, as directed by the injector's hand.
2) The integrity of the fibro-fatty tissue network holds the septal mucosa tightly together, preventing lateral expansion of the membranous mucosa under the pressure of the filler bolus. Because of this, and the septal cartilage being fixed, the inflated “bounce house” effect can only lead to the rising or elongation of the “free” nasal tip structures.

### EXAMPLES

In the examples below, the patients' ages spanned from 18 to 84, with most of the patients in the range of 25 to 60. There were 17 male patients included in this study. Among the Asian patients, 78 patients only required pure tip elevation effect and 35 needed only tip elongation. The majority needed both tip elevation and elongation at the same time (674 treated with the first injection technique described above (also referred to as “tip-lift injection technique), 156 treated with the second injection technique described above (also referred to as “tip-elongation technique”), and 189 with a stacking of both techniques). The need for both tip elevation and elongation effects was demonstrated even in many patients with relatively long noses because after tip lifting, the cephalad rotation visually shortens the noses, requiring simultaneous tip elongation for more aesthetically pleasing results. The treatment time was 2 to 10 minutes for a nose makeover. Depending on the patients, the amount of filler used was from 0.4 to 0.8 cc for either nose tip-lift technique or nose tip-elongation technique. The filler used was Restalyne® Lyft (Galderma). The nose tip was elongated by 2 mm to 6 mm (mean value 3.13 mm, measured from the rhinion to tip defining points), and the nose tip was elevated by 2 mm to 8 mm (mean value 5.07 mm, measured from the tip to the nasolabial junction). Yet, even in those patients with limited tissue expandability, when serial injections of the nose tip were carried out, the nasal tip elevation and elongation was further improved. Hence, serial injection seemed to be a useful tool for “tissue expansion” with the filler in the septal space over time

### Example 1

### Method of Nose Tip-Lift Injection Technique

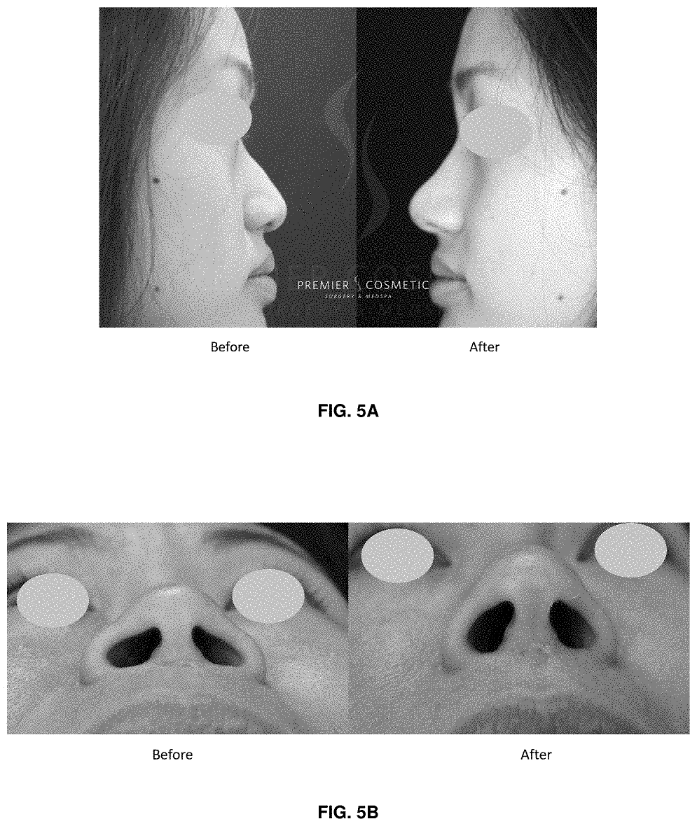

This example demonstrates the treatment effect of a patient with significant nasal tip deficiency. An amount of 0.6 cm³ of filler was injected in the tip region with the tip-lift technique and 0.4 cm³ for the radix and the canine fossa. As shown in FIG. 5A, this patient had great improvement in tip projection, nasolabial angle, and overall profile. Even the large alar (see alar 40, FIGS. 2A and 2D) structure looks much smaller and less prominent. The infra-tip lobule to columella ratio was improved. The pouting lips rest in a better position in relationship to the nasal structure. With a little enhancement of the radix, the whole nose looks improved. The worm's eye view of another patient (received 0.55 cm³ of filler was injected for tip elevation, 0.15 cm³ for the radix, 0.15 cm³ for the dorsum, and 0.15 cm³ for the supra tip region) shown in FIG. 5B shows that: 1) the nasal tip was elevated; 2) the dome stands out, showing fine tip cartilaginous structures; 3) the infra-tip lobule was more in proportion to the whole nasal structure; 4) the footplates were elevated; 5) the columella was lengthened; 6) the nostrils were elongated and more upright; 7) the alar width was reduced; 8) the dome looks somewhat narrower; 9) surprisingly, the philtrum columns, as well as the cupid's bow became more prominent.

The nasolabial angle improvement came naturally with the distention of the lower part of the septal space between the caudal septum and the columella. With the tip raised from below, the nasal skin and soft tissue became tense, pressing on the tip cartilaginous structures and making them more compact together, showing better tip defining points. Additionally, because the tip was elevated without the piling on of filler on the top, the tip remained natural, while the columella was lengthened. Subsequently, the infra-tip lobule length to columella length ratio was better. Next, the nostril was changed to a better shape, becoming oblong and standing up, instead of being low lying and flat. The alar width was narrowed as a result of the gathering of tissue more centrally. After all these changes, the nose became smaller from the frontal view.

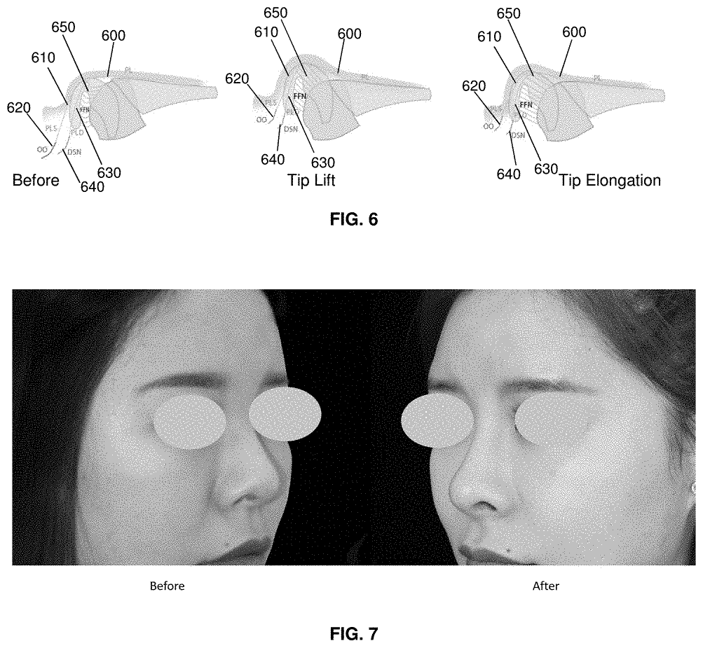

Note that the philtrum columns became more prominent, the upper lip became shorter, and hanging nose was corrected with this simple tip lift injection. Aging is accompanied by effacement of philtrum prominence and upper lip lengthening. Correcting these features was very welcoming in aging patients. Daniel et al. described their findings in the study of human nasal base anatomical structures and analyzed in detail as to the functionality and the possible inter-play of these previously ignored structures. Daniel, R. K., et al., The Lower Nasal Base: An Anatomical Study Aesthetic Surgery Journal 2013; 33:222-232. More in-depth studies were done to dissect the nasal ligaments and relevant structures. Daniel, R. K., et al., The Nasal Ligaments and Tip Support in Rhinoplasty: An Anatomical Study Aesthetic Surgery Journal 2018, Vol 38(4) 357-368. A diagram shown in FIG. 6 may explain how the hanging nose is prevented from being further pulled down when smiling and why the philtrum columns are enhanced. As shown in FIG. 6, the Pitanguy ligament 600 (PL, the blue lines) superficial branch 610 (PLS) connects directly with the orbicularis oris 620 (the anterior red stripe, OO), and the deep branch 630 (PLD), with the depressor septi nasi 640 (the posterior red stripe, DSN). When these structures are tensed up with filler injection in the space between the caudal septal cartilage and the columella, the ligaments along with the attached musculatures are all affected. The abundant thick fibro-fatty tissue network 650 (FFN) is also depicted in FIG. 6. The FFN becomes stretched after a bolus of filler is injected into this space (for tip lifting or tip elongation). The abundant FFN in the septum space may be responsible for holding the injected filler and therefore, maintaining the inflated “bounce house” effect. After the tip is lifted or elongated, the resulting tension from the tensed-up PL may be responsible for suppression of the function of depressor septi nasi (DSN) and shortening of upper lip (through pulling on the orbicularis oris, OO). The combined passive pull on the depressor septi nasi and orbicularis oris, in turn may make the philtrum columns more prominent, and as a result, there is a better upper lip positioning (with the help of canine fossa and anterior nasal spine augmentation). Finally, as the tip is lifted or elongated, the depressor septi nasi is pulled and tensed up to the extreme, thus losing the ability to contract. This may explain why the treated patients with hanging noses completely lack downward pulling of the tip when smiling.

### Example 2

### Method of Nose Tip-Elongation Injection Technique

This example demonstrates the treatment of patients with significant nasal tip length deficiency. An amount of 0.7 cm³ of filler was injected in the tip region with the tip-elongation technique and 0.3 cm³ in the radix and the glabella region to better connect the nose with the brow structures. The effects show both tip elevation and elongation. Another 1 cm³ of the filler was injected in the canine fossa area. FIG. 7 shows the result of a typical tip elongation result. The tip definition, height and nasal labial angle were also enhanced. Further, the upper lip length was shortened, with a better philtrum/columella relationship. This patient demonstrates better correlation of the eyes, the nose, the brows and the upper lip, forming a better-looking aesthetic unit.

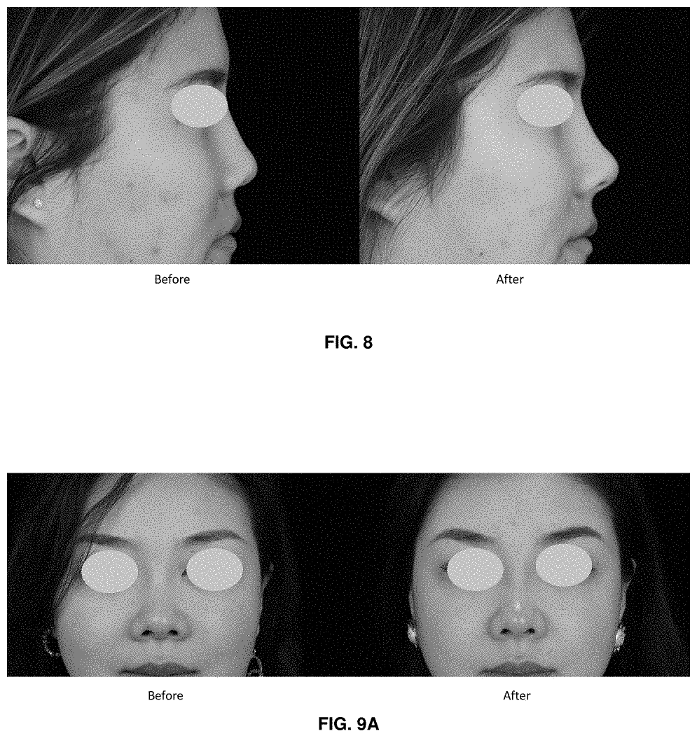

In another patient, 0.8 cm³ of filler was used for injection with the nose tip-elongation technique and 0.2 cm³ w of filler as used for canine fossa augmentation. The results are shown in FIG. 8 showing the nose was elongated along with correction of veiling. Also notable is the significant shortening of the upper lip. This patient's profile was significantly improved at the same time.

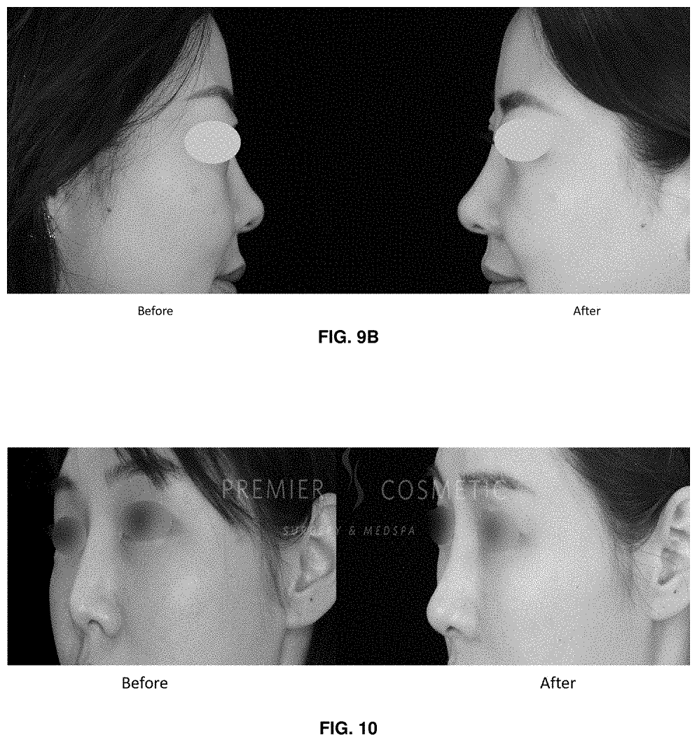

Shown in FIGS. 9A and 9B is a patient who had both tip-lift and tip-elongation. An amount of 0.4 cm³ of filler was used for injection with the nose tip-lift technique and 0.3 cm³ of filler for injection with the nose tip-elongation technique. The leftover 0.3 cm³ of filler was used for canine fossa augmentation. It was apparent that the patient's tip defining points became closer, and alar width narrower, and the relationship between the philtrum columns and the columella became tighter in FIG. 9A. In FIG. 9B, the tip lift and elongation are seen more clearly, with significant upper lip shortening, the nasolabial angle was better, and the facial profile was nicely balanced. Interestingly, the tip lifting/elongation also brought changes to the relationship of philtrum columns to the columella, which is aesthetically pleasing. While rarely is this tightly knit relationship seen in Asians, it is a normalcy in most Caucasians.

Bolus injection into the middle of the potential septal space between the caudal septum and the columella can generate both lifting and elongation effects of the tip, albeit the elongation effect is modest. When more elongation is desired, this nose tip-elongation technique is employed, with the needle inserted in a more cephalic position on the nasal tip skin, hugging the caudal septal cartilage. The closer the needle to the septal cartilage, the better the elongation effect.

The same is true when nose tip-elongation injection is performed. Some degree of tip lifting effect is also achieved, and this effect is controllable with the injector's non-dominant hand, by manipulating the pressure on the nose tip. Regarding nose tip elongation: 1) it can be a difficult task to elongate a nose, even with rib cartilage for surgical rhinoplasty; and 2) the current study involves more than 1000 cases that demonstrated effectiveness of nose elongation in most of them.

### Example 3

### Method of Nose Tip-Elongation and Tip-Lift Injection Techniques for Small Noses

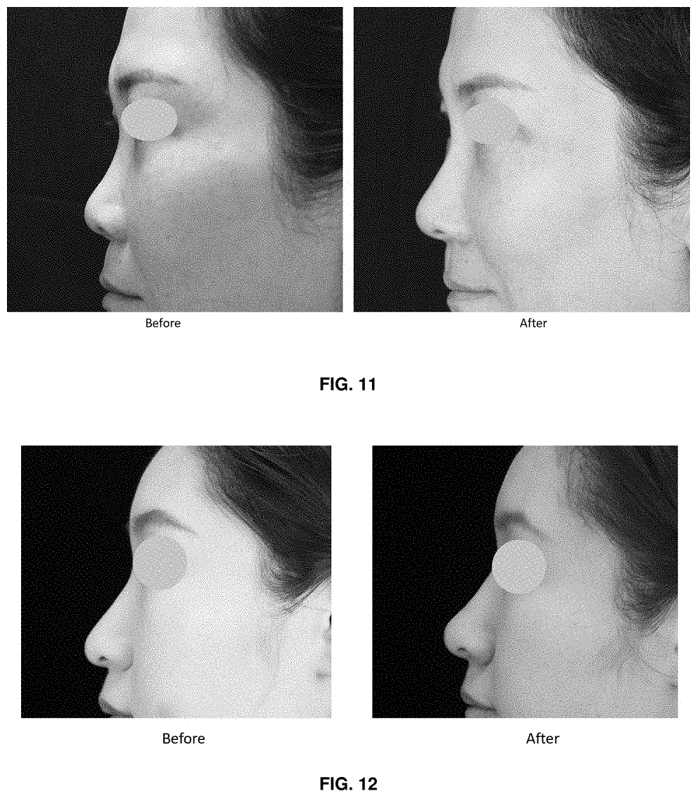

This example demonstrates the treatment of patients with significant “small nose” deficiency. Extremely small noses pose a unique challenge to the treating physicians, because the heights of the tip, the dorsum and the radix all need to be corrected at the same time. With conventional filler injection methods (augmentation of the anterior nasal spine, creation of columella strut with a filler, and/or sometimes, augmenting the tip with a filler), due to the limitation in achieving tip elevation height, these patients typically could only obtain limited aesthetic results. However, with the nose tip-lifting and nose tip-elongation method disclosed herein, it becomes much easier because the filler injection with the two techniques can be stacked. The patient in FIG. 10 received 0.6 cm³ of filler for tip elevation with the nose tip-lift technique, 0.6 cm³ of filler for tip elongation with nose tip-elongation technique, 0.1 cm³ of filler for the supra-tip region, 0.2 cm³ of filler for tip enlargement by injecting in between the domal cartilages, and 0.5 cm³ of filler for the canine fossa augmentation. The patient in FIG. 11 received 0.6 cm³ of filler for tip lift with the nose tip-lift technique, 0.4 cm³ of filler for tip elongation with the nose tip-elongation technique, 0.4 cm³ of filler for dorsum and radix injection, and 0.6 cm³ of filler for canine fossa augmentation. Both patients' facial balance was corrected (FIGS. 10 and 11).

### Example 4

### Method of Nose Tip-Lift Injection Techniques for Long Noses

This example demonstrates the treatment of a patient with significant long nose deformity. Due to the relatively lower tip height of the Asian noses, most of the long noses can be manipulated with the tip injection to make them shorter by up turning the tips. The injection was focused on lifting the tip and at the same time, the injector's non-dominant hand pushed the whole tip structure and the upper half of the columella in a cephalic direction. A total volume of 0.8 cm³ of filler was injected for injection with the nose tip-lift technique and 0.2 cm³ of filler was injected in the canine fossa. As shown in FIG. 12, the patient's nose shortened substantially with the upturning of the tip, resulting in correction of not only the nasal length, but also the nasolabial angle, the alar to tip ratio, the length of the columella, and the nasal tip height to nasal length ratio. Treatment of long noses in Caucasians and Hispanics was also quite successful.

### Example 5

### Method of Nose Tip-Lift Injection Techniques for Hanging Noses

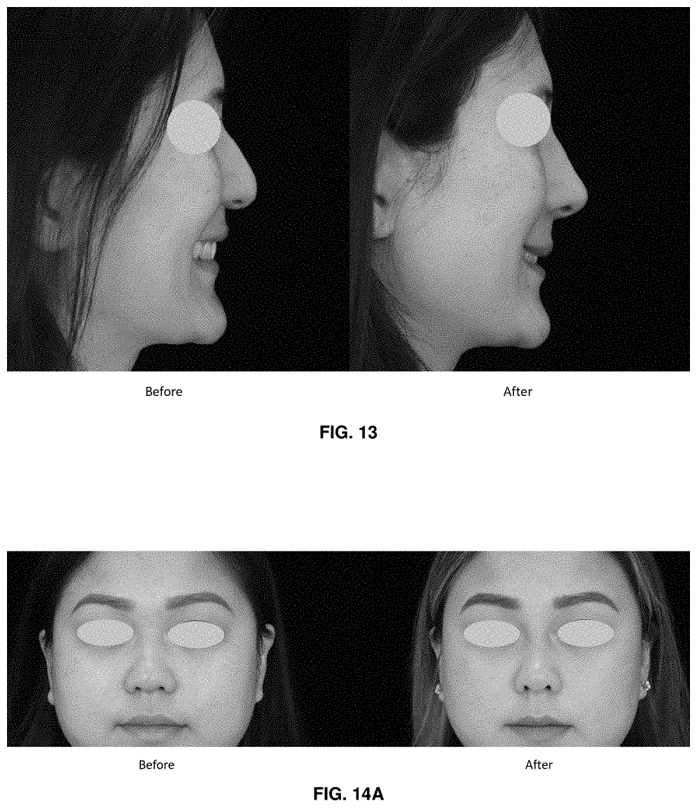

This example demonstrates the treatment of a patient with significant hanging nose deficiency. These patients demonstrate hanging noses, especially when smiling, causing the nose tip height to be pulled even lower, with further narrowing of the nasolabial angle. The culprit worsening the nose hanging effect is the activation depressor septi nasi muscles. This effect was corrected by the tip lift injection method using 0.7 cm³ of filler and 0.3 cm³ of filler was delivered in the canine fossa, as shown in FIG. 13. Also see FIGS. 5 and 6 in Example 1 for further discussions on the treatment mechanisms.

### Example 6

### Method of Nose Tip-Lift Injection Techniques for “Large” Noses

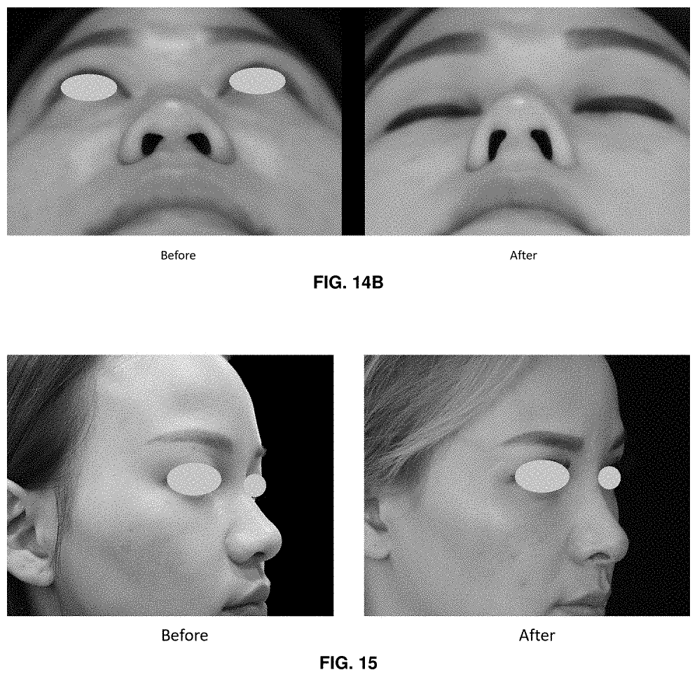

This example demonstrates the treatment of patients with a significant large nose deficiency. These patients typically have a flat, round and bulbous tip, with wide alar widths. A total volume of 0.8 cm³ of filler was injected for injection with the nose tip-lift technique, 0.2 cm³ of filler was injected in the radix, and 1 cm³ of filler was injected in the canine fossa. FIG. 14A shows that after tip lift, the domal structure became smaller, the alar width narrower, making it more in proportion with the eyes and the lips. The tip defining points became clearer and the nose was slimmed down. The worm's eye view (FIG. 14B) of the nose demonstrated more substantial changes. It shows better infra-tip lobule/columella ratio, elevated footplates, and elongated nostrils. Not surprisingly, the nose appears much smaller. In FIG. 15, the patient had two tip injections separated by 6 months. The first injection was 0.6 cm³ of filler for injection with the nose tip-lift technique and 0.4 cm³ of filler for the dorsum and radix. For the second injection, 0.3 cm³ of filler was injected with the nose tip-lift technique, and 0.7 cm³ of filler was injected for the canine fossa. Another 1 cm³ of filler was used for a brow ridge augmentation to achieve a “Double Ogee” effect. Ramirez O M. Double ogee facial rejuvenation. Cirugia Plastica Ibero-Latinoamericana. 2007; 33:1-14. FIG. 15 shows clearly that the alar cartilage became much less prominent. The nose also became longer with the simple tip lift injection. Even the size of the alar appears much smaller, demonstrating a better alar to tip ratio. Veiling was also improved. Further, the upper lip appears shorter and better poised.

### Example 7

### Method of Nose Tip-Lift and Tip-Elongation Injection Techniques for Nasal Humps

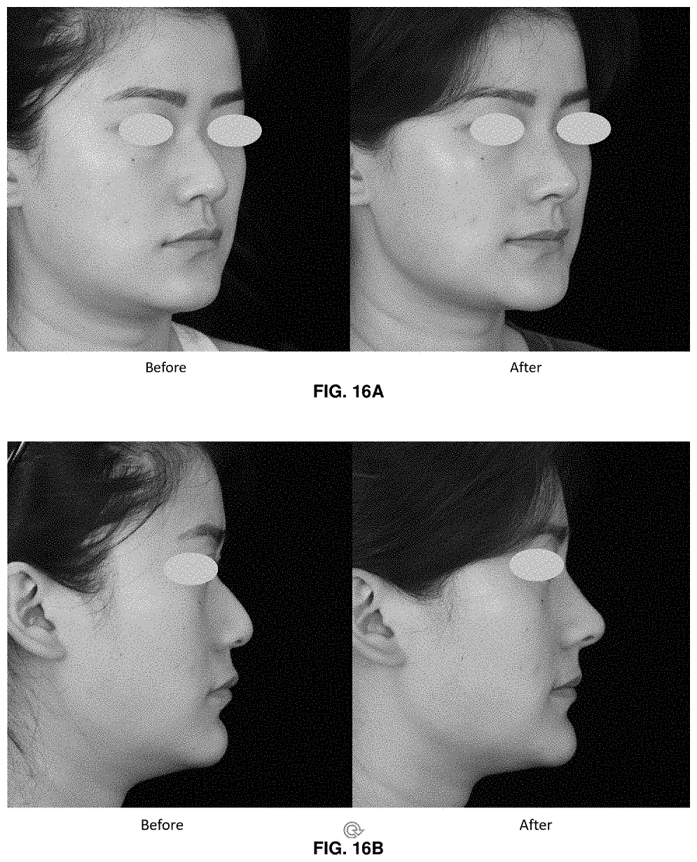

This example demonstrates the treatment of patients with significant nasal hump deficiency. In the past, most of these patients would have required surgical correction. But the current effective nose tip-lift and tip-elongation injection techniques make it possible for most of these patients to avoid surgeries. The patient in FIGS. 16A and 16B received 0.6 cm³ of filler injected with the nose tip-lift technique (with some elongation, made possible with the pulling on the columella, while injecting), 0.3 cm³ of filler for radix improvement, 0.2 cm³ of filler in the glabella region, 0.05 cm³ filler each for augmenting the medial brow heads (with the injection done sub-dermally), and 0.8 cm³ of filler was used for canine fossa improvement. The before image in FIG. 16A shows a tall hump and a short nose. The after image in FIG. 16A shows complete correction of the radix and the tip with satisfactory camouflage of the nasal hump. The tip definition was also improved. Also note that the change of philtrum columns in relationship to columella, fusing the two seemingly unrelated structures into one natural aesthetic unit. The upper lip was shortened, and better positioned. FIG. 16B of the same patient further demonstrates a newly formed supra-tip break, with well-defined nasal tip. From this angle, the upper tip shortening, and the repositioning of the lip structures are more visible. Further, the patient shows a more elegant nasolabial angle, the profile and facial proportion are considerably improved. The eyes become brighter.

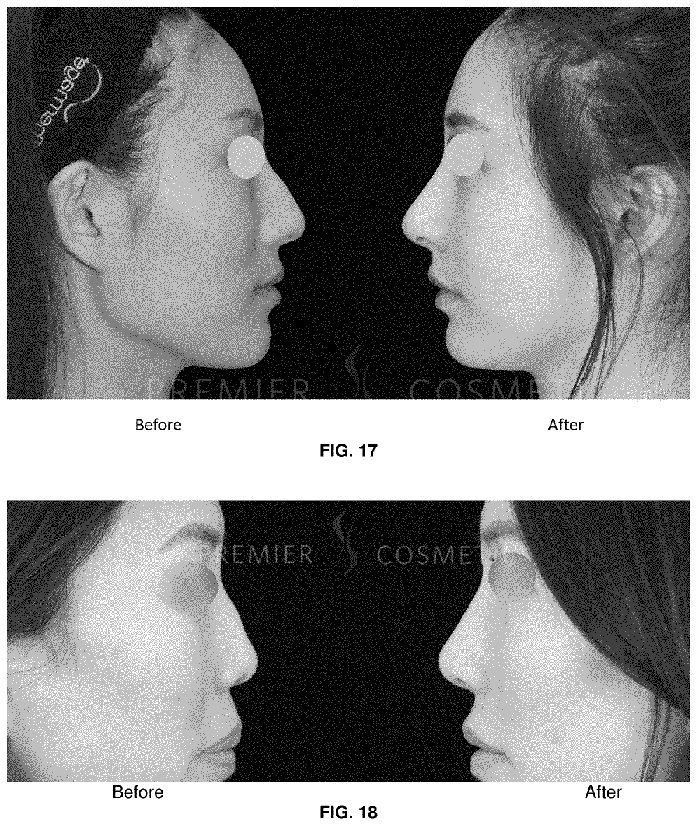

The patient in FIG. 17 received 0.6 cm³ of filler for injection with the nose tip-lift technique, 0.2 cm³ of filler for injection with the nose tip-elongation technique to prevent over-rotation of the nose tip, 0.2 cm³ filler for the radix area, 0.1 cm³ of filler for the supra-tip area to camouflage the hump structure, and 0.1 cm³ of filler was used for canine fossa augmentation. Another 1 cm³ of filler was used for brow ridge and glabella augmentation. The before image in FIG. 17 shows a patient with a high hump and relatively long nose. After evaluating this patient, the plan was to elevate the nose tip, minimize the rotation of the tip by modestly elongating the tip after tip elevation, and at the same time, the filler was used to augment the radix. Because the hump was much higher than usual, the brow ridges were augmented to justify the final height of the nasal structure and to keep the starting point of the nose root low, so this patient did not end up having an “Avatar” look. In the after image in FIG. 17, the upper lip was shortened and better positioned. This patient also had severe gummy smile prior to the treatment. Surprisingly, with this single tip lift injection (along with canine fossa augmentation) session, the gummy smile was cured (picture not shown).

### Example 8

### Method of Nose Tip-Lift and Tip-Elongation Injection Techniques for Saddle Nose Deformities

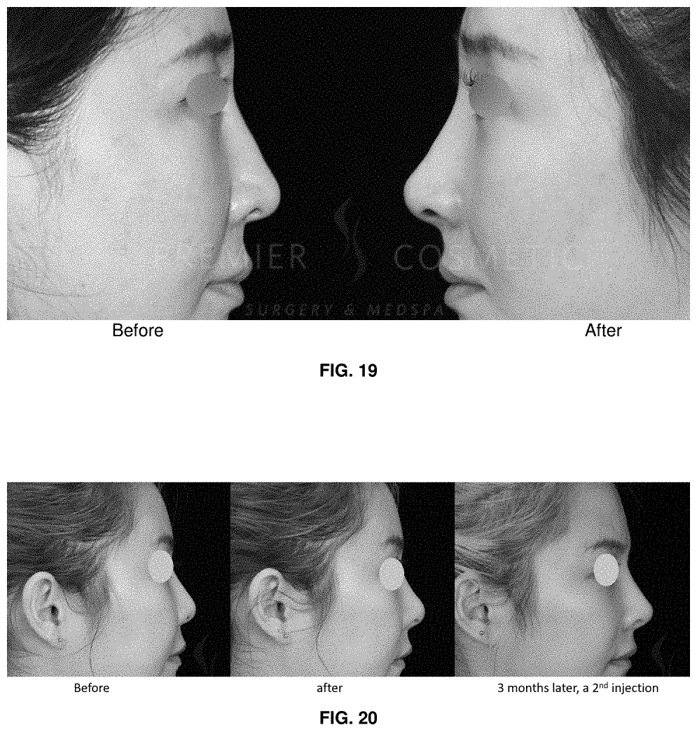

This example demonstrates the treatment of patients with saddle nose deformities. There are plenty of publications describing the treatment of saddle nose deformity using fillers. However, most of these treatment modalities are focused on camouflaging the saddle deformity alone. In most of these deformed noses, covering up the deformity alone is certainly not enough, due to the profound accompanying tip deficiency. The patient in FIG. 18 received 0.4 cm³ of filler for injection with the nose tip-lift technique, 0.2 cm³ of filler for injection with the nose tip-elongation technique, 0.15 cm³ of filler for radix augmentation, 0.25 cm³ of filler for the saddle deformity area, and 1 cm³ of filler was injected in the canine fossa area. The patient in FIG. 19 received 0.6 cm³ of filler for injection with the nose tip-lift technique, 0.2 cm³ of filler for radix and dorsum improvement, 0.2 cm³ of filler for the saddle deformity area injection, and 1 cm³ of filler was used for canine fossa augmentation. Both FIGS. 18 and 19 shows saddle nose deformity along with tip lift and/or elongation with good profile improvement. Note that in both cases, the upper lips were much shortened and better positioned. In essence, correcting saddle nose deformity is better done with the whole nasal structure considered for better outcomes.

### Example 9

### Method of Obtaining Progressive Changes with Nose Tip-Lift and Tip-Elongation Injection Techniques

This example demonstrates the treatment of patients with serial injections in the nasal tip to obtain progressive improvements. Often, there is the insufficiency of soft tissue envelop when a significant improvement is desired. Additionally, the mobility of the tip could be inadequate. After the first injection, the swelling usually settles by 1 week. In the following few weeks, some of the fillers will dissipate into the surrounding tissues, losing some supporting effect. As a result, patients are asked to come back for a second enhancement at 3 weeks to 3 months. During this second injection (or second enhancement or treatment), the filler used for tip may only be half as much. It is after this second enhancement, the result may become more stable. The third enhancement may be at a 9-month to 1 year interval.

The patient in FIG. 20 had two consecutive injections in two months. The first treatment was 0.7 cm³ of filler for injection with the nose tip-lift technique (with modest elongation, manipulated with the injector's non-dominant hand) and 0.3 cm³ of filler for the radix and dorsum. The second treatment was 0.4 cm³ of filler for injection with the nose tip-lift technique, 0.3 cm³ of filler for injection with the nose tip-elongation technique, 0.3 cm³ of filler for the radix and dorsum, and 1 cm³ of filler for the canine fossa. FIG. 20 shows good tip improvement after the first injection session. Three months later, the patient's nose tip became mobile again. The second treatment evidently confirmed that the filler in the septal space did exert a desired “tissue expansion” effect.

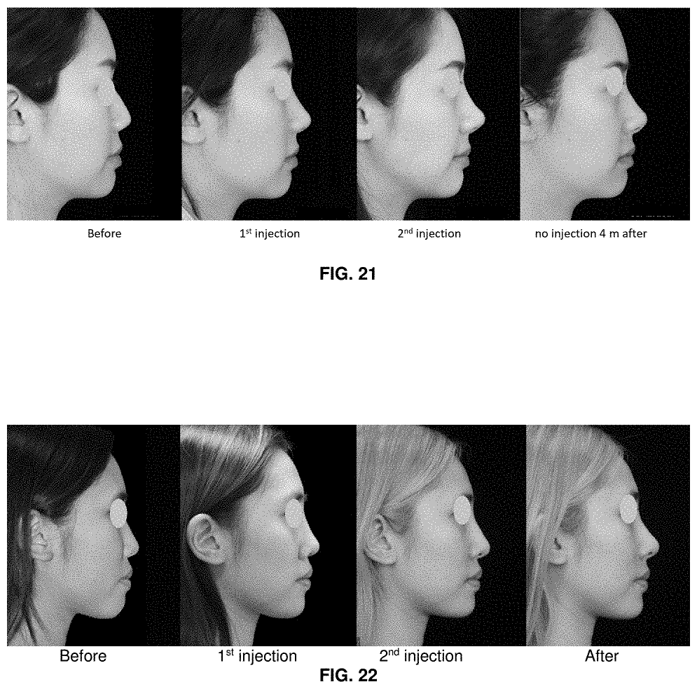

The patient in FIG. 21 had two consecutive injections 4 weeks apart. The first treatment was 0.8 cm³ of filler for injection with the nose tip-lift technique, 0.3 cm³ of filler for injection with the nose tip-elongation technique, 0.3 cm³ of filler for the radix and glabella and brow head augmentation, 0.6 cm³ of filler was used for canine fossa improvement. The second treatment was 0.4 cm³ of filler for injection with the nose tip-lift technique, 0.1 cm³ of filler for injection with the nose tip-elongation technique, 0.1 cm³ of filler for the radix, and 0.4 cm³ of filler for the canine fossa. FIG. 21 shows significant deficiency in nose projection in the before image. The radix was also too low. Furthermore, there was veiling of the columella. After the first injection, the patient's profile improved immensely. In the second treatment, emphasis was placed on improving the tip further. However, the tip elongation effort was a failure. Only 0.1 cm³ of filler was deposited for this purpose. Consequently, this patient exhibited over-rotation of the tip. Despite this, the patient liked the overall result. Four months later, the tip over-rotation settled and a shapely supra tip break is evident. The patient's profile was improved with better lip positioning.

Oftentimes, the filler improvement of a nose is significantly limited by the availability of soft tissue envelop. Interestingly, a “tissue expansion” phenomenon was observed. FIGS. 20 and 21 both demonstrate how effective this “tissue expansion” effect works. When sequential injections are applied, the septal space expands over time, enabling the practitioner to further improve the nose. On this note, it is noteworthy that cross-linked hyaluronic acid could induce neo-collagen formation. In any case, the “tissue expansion” effect of a filler injected into the septal space could be utilized to expand the nose tip such that the resulting expanded nose could be beneficial for subsequent rhinoplasty surgeries when tissue envelope availability is a concern.

### Example 10

### Results Showing the Longevity with the Methods of Nose Tip-Lift and Tip-Elongation Injection Techniques

This example demonstrates the longevity of filler with the methods of nose tip-lift and tip-elongation injection techniques. The patient in FIG. 22 received two injections a year apart. The first treatment was 0.6 cm³ of filler for injection with the nose tip-lift technique, 0.2 cm³ of filler for injection with the nose tip-elongation technique, 0.2 cm³ of filler for glabella and medial brow head augmentation, and 0.5 cm³ of filler for the radix and dorsum improvement. Because of the tissue limitation, with both tip-lift and tip-elongation techniques, the injections were stopped after a total of 0.8 cm³ of filler was injected in the tip. Also, 0.5 cm³ of filler was used for canine fossa, and 1 cm³ of filler was injected in the chin for augmentation of the pogonion. The second treatment became easier after waiting for one year, when the tip became more mobile. In the second treatment, 0.5 cm³ of filler was used for injection with the nose tip-lift and tip-elongation techniques and 0.5 cm³ of filler for canine fossa improvement. FIG. 22 shows a patient with concave facial feature, nasal height insufficiency, narrow nasolabial angle and flat radix. The upper lip pouting was quite evident. A careful examination shows lateral labial incompetence. After the first treatment, this labial incompetence was corrected. One year later, the nasal improvement remains, except for the tip becoming slightly lower. Yet, the columella kept improving over time. The upper lip remained shorter. Interestingly, the lateral labial incompetence showed again. This defect was corrected with the second injection of fillers, along with better tip projection, better nasolabial angle and better lip positioning.

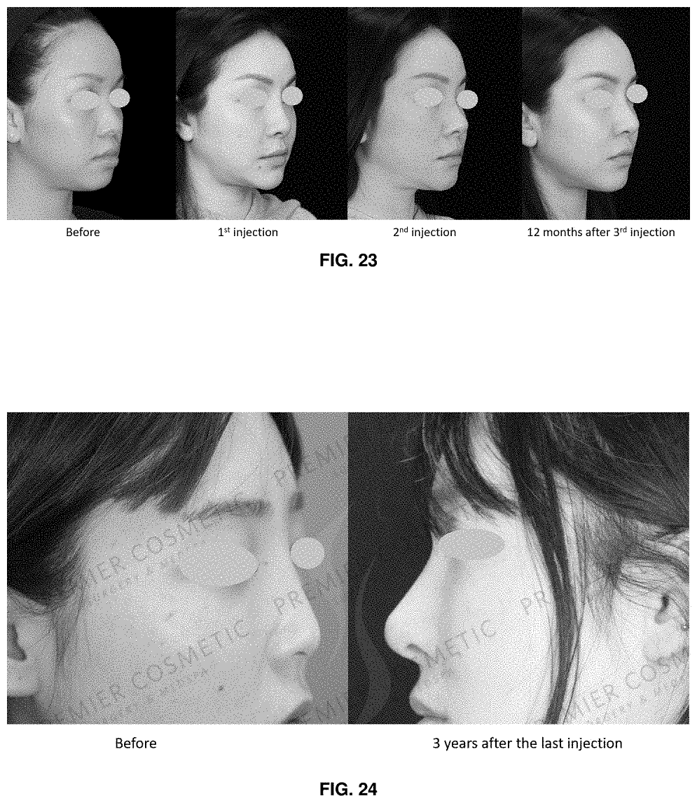

The patient in FIG. 23 received three injections. The first treatment was 0.5 cm³ of filler for injection with the nose tip-lift technique, 0.3 cm³ of filler for injection with the nose tip-elongation technique, 0.6 cm³ of filler for radix, dorsum, brow ridge and glabella improvement, 1 cm³ of filler was used for canine fossa augmentation, and 1.4 cm³ of filler for chin augmentation. The second treatment, performed 2 months later, was 0.4 cm³ of filler for injection with the nose tip-lift technique, 0.2 cm³ of filler for injection with the nose tip-elongation technique, and 0.4 cm³ of filler for canine fossa augmentation. Also, 1 cm³ of filler was used for cheek improvement. The third treatment was done after 10 months after the second injection with 0.3 cm³ of filler for injection with the nose tip-elongation technique, 0.1 cm³ of filler for glabella, 0.1 cm³ of filler for brow head, 0.1 cm³ of filler for radix, and 0.4 cm³ of filler for the canine fossa. FIG. 23 shows the longevity of nose tip filler injection, and the importance of filler injection in the surrounding areas for the achievement of a better aesthetic result regarding facial profile improvement. Note that after the nasolabial angle/veiling was improved and with the aid of canine fossa (nasal base) enhancement, the upper lip was better positioned, with the potential of not only correcting the duck bill looks (due to narrow nasolabial angle and possibly short chin), but also amending the labial incompetence.

Again, FIGS. 22 and 23 both demonstrated how effective this “tissue expansion” effect works. When sequential injections are applied, the septal space expands over time, enabling the practitioner to further improve the nose.

The patient in FIG. 24 received a series of five injections. For the first treatment, an injection with a Vycross® filler was performed for tip and canine fossa improvement. This was followed by using a NASHA filler for the second and third treatments on the dorsum and mostly for the tip lifting and elongation. Each time, 1 cm³ of filler was used. Two more injections with a NASHA gel were performed in the following 18 months. Three years later, the nose remained about the same as the last time after the injection. In FIG. 24, this patient presented a very difficult situation. It took 5 sessions of injection to achieve a very satisfactory result. Surprisingly, this patient had a very enduring result after a two-year long treatment packed with 5 injection sessions. Further analysis of the case in FIG. 24 suggests that the initial injection of a Vycross® filler may have helped the longevity of this patient's nose enhancement result, because of the long staying effect and the water absorbing capacity. It is believed that the combination of a Vycross® filler and a NASHA filler that made this patient's nose enhancement lasting much longer than expected. Indeed, FIG. 24 suggests that this type of filler distention induced “tissue expansion” might become almost “permanent.”

### Example 11

### Results Showing the Effectiveness with the Methods of Nose Tip-Lift and Tip-Elongation Injection Techniques for Contracted Short Noses Troubled with a Nasal Implant

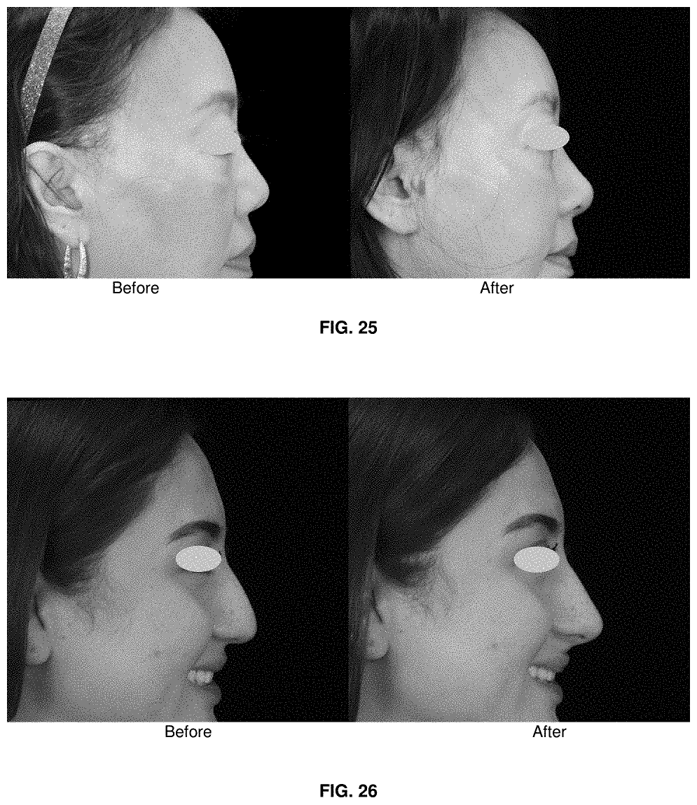

The patient in FIG. 25 had a silicone nasal implant (“L” shaped) 30 years prior to treatment, and because of capsular contracture, the nose became shorter and shorter with resulting severe deformity. First, the patient received 1 cm³ of filler injected into the space behind the short arm of the “L” shaped silicone implant, by using a needle to pierce through the domal part of the nasal skin and the implant wherein the nose became longer, and the nasolabial angle was much improved. Then 0.7 cm³ of filler was used for nose elongation, and 0.3 cm³ of filler for nose tip elevation. After injecting the nose tip with both the nose tip-elongation and nose tip-lift techniques, the patient's deformity was completely corrected, with the “L” shaped implant being pushed in both downward and upward directions.

### Example 12

### Results Showing the Effectiveness with the Methods of Nose Tip-Lift and Tip-Elongation Injection Techniques in Other Races

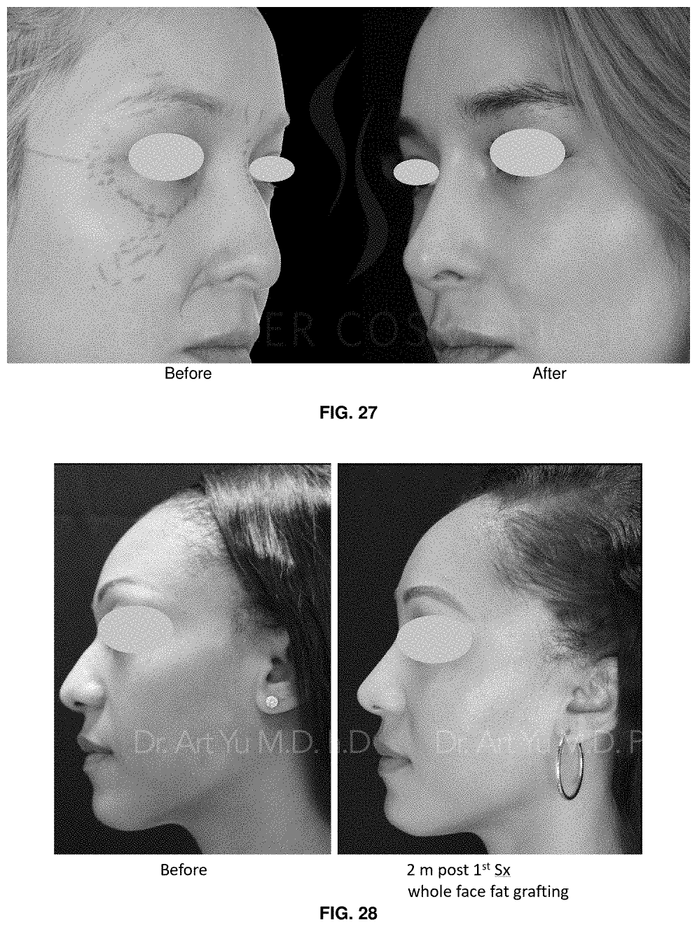

Other races can also be satisfactorily treated with the aforementioned nose tip-elongation and nose tip-lift injection methods. Shown in FIG. 26 is a patient with a typical hanging nose tip deformity, which was cured with a simple nose tip-lift injection technique. The patient in FIG. 26 received 0.85 cm³ of filler for injection with the nose tip-lift technique and 0.15 cm³ of filler for the radix improvement such that the hanging nose deformity along with the down-pulling of the tip while smiling were cured. FIG. 27 shows a patient with hanging nose tip deformity, complicated with low radix and a high hump. The patient in FIG. 27 was successfully treated with a combination of autologous fat grafting for the upper forehead, temples, tear troughs, midface, radix, and the nasolabial folds, and a nose tip injection with a NASHA filler using the nose tip-lift injection technique depositing 0.8 cm³ of filler in the septal space. FIG. 28 is a patient with general loss of volume of the whole face, along with a low radix, high hump and hanging nose deformity. The treatment was accomplished with whole face fat grafting and sculpturing. The tip was successfully treated with autologous fat grafting using the nose tip-lift technique with 1 cm³ of fat injected into the septal space and 0.3 cm³ of fat injected into the supra-tip region, using an 18G blunt cannula. Also, 1 cm³ of fat was injected into the canine fossa area for further improvement.

All publications, patent applications, issued patents and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

What is claimed is:

1. A nonsurgical method of modifying at least a portion of a nose of a subject, the method comprising:
performing one or more of:
(i) a first injection of a first filler composition comprising:
depositing the first filler composition in a first region defined as a septal space behind a middle crura and a medial crura of the nose, substantially level and/or above an anterior nasal spine of the nose, and in front of a caudal septal cartilage of the nose; and
(ii) a second injection of a second filler composition comprising:
depositing the second filler composition in a second region defined as the septal space behind the middle crura and medial crura and substantially level with footplate of the nose and the columella segment of the medial crura of the nose.

2. The method of claim 1, wherein the modifying comprises elevating a tip of the nose, elevating the columella of the nose, elongating the columella of the nose, elongating the tip of the nose, shortening the tip of the nose, increasing firmness of the nose, narrowing of a dome of the nose, elevating the footplates, decreasing width of an alar of the nose, increasing prominence of philtrum columns, increasing oblong shape of nostrils, or any combination thereof.

3. The method of claim 1, wherein first injection further comprises holding a columella of the nose, a nasal tip of the nose, or both in a first direction to extend open the septal space and/or the second injection comprises holding a columella of the nose, a nasal tip of the nose, or both in a second direction to extend open the septal space.

4. The method of claim 3, wherein the first direction is selected from the group consisting of an upward direction, a caudal direction, a rotated direction, and a combination thereof and/or the second direction is a distal direction, a caudal direction, an upward direction, or a combination thereof.

5. The method of claim 4, wherein the first direction is:
(i) the upward direction and the nasal tip and columella are pressed against anterior septal cartilage of the nose and a tip of the nose is elevated;
(ii) is the caudal direction and a tip of the nose is elevated, elongated, or both; or
(iii) is the upward direction and the rotated direction and a tip of the nose and/or the nose is shortened.

6. The method of claim 4, wherein the second direction is a distal direction, a caudal direction, an upward direction, or a combination thereof.

7. The method of claim 1, wherein one or more of the following are satisfied:
(i) the second filler composition is deposited in front of the anterior nasal spine;
(ii) the first injection, the second injection, or both deposit the first filler composition, the second filler composition, or both prior to the first injection, the second injection, or both reaching the anterior nasal spine;
(iii) first filler composition, the second filler composition, or both are deposited in the septal space of the nose; and
(iv) the first filler composition is not deposited in a surface of a tip of the nose.

8. The method of claim 1, wherein each of the first filler composition and the second filer composition is deposited with a needle or a cannula.

9. The method of claim 1, wherein the first injection, the second injection, or both comprise a retro-injection or an antegrade injection and/or an amount of each of the first filler composition deposited and the second filler composition deposited is about 0.01 cm³ to about 2 cm³.

10. The method of claim 1, wherein the first injection, the second injection, or both enter the nose in one or more of the following areas: a tip region of the nose, between a middle crura, an infra-tip lobule, a supra-tip area, the columella region, an intra-nasal region, or an intra-oral region.

11. The method of claim 1, wherein a tip of the nose is elongated by greater than 0 mm to about 10 mm and/or the tip of the nose is elevated by greater than 0 mm to about 10 mm.

12. The method of claim 1, wherein the tip of the nose is not elevated and/or not elongated.

13. The method of claim 1, wherein the first filler composition and the second filler composition each comprises hyaluronic acid, calcium hydroxyapatite, autologous fat, heterologous fat, soft tissue, cartilage, bone, agar, agarose, collagen, dextran beads, polymethyl methacrylate (PMMA), methyl methacrylate, polylactic acid, bone powder of any origin, polydioxanone (PDO), silicone, polytetrafluoroethylene (PTFE), expanded polytetrafluoroethylene (ePTFE), a polyalkylimide, silk, human tissue, animal tissue, plant substrate, synthetic material or a combination thereof.

14. The method of claim 1, wherein the first filler composition and the second filler composition each are selected from the group consisting of a nonanimal stabilized hyaluronic acid (NASHA), a cross-linked nonanimal derived hyaluronic acid gel, an animal derived hyaluronic acid, a non-cross-linked hyaluronic acid product, and a combination thereof.

15. The method of claim 1, wherein the first injection, the second injection, or both are performed more than once and/or both the first injection and the second injection are performed.

16. The method of claim 15, wherein the first filler composition and the second filler composition are the same.

17. The method of claim 15, wherein the first filler composition and the second filler composition are different.

18. The method of claim 1, further comprising performing a third injection of a third filler composition comprising depositing the third filler composition in the radix region of the nose, a dorsum region of the nose, or both.

19. The method of claim 1, wherein the subject has one or more of: a nasal hump deformity, a saddle nose deformity, a nasal deformity caused by capsular contracture after a nasal implant.

20. The method of claim 1, wherein the subject is Asian, Caucasian, Hispanic, Arabian, African American, or a combination thereof.
